(19) United States
(12) Patent Application Publication
(10) Pub. No.: US 2011/0020439 A1
(43) Pub. Date: Jan. 27, 2011

(54) DELAYED RELEASE COMPOSITIONS OF DULOXETINE

(76) Inventors: Shrenik Annasaheb Kole, Maharashtra (IN); Ashok Kumar Sahoo, Maharashtra (IN); Makarand Krishnakumar Avachat, Maharashtra (IN)

Correspondence Address: FULBRIGHT & JAWORSKI L.L.P.;Attn: MN IP Docket, 600 Congress Avenue, Suite 2400, Austin, TX, 78701, US

(21) Appl. No.: 12/934,148

(22) PCT Filed: Mar. 17, 2009
(86) PCT No.: PCT/IN09/00183

§ 371 Date: Sep. 23, 2010

(30) Foreign Application Priority Data
Mar. 24, 2008 (IN) 580/KOL/2008

(51) Int. Cl. A61K 31/381; A61K 9/52; A61K 9/22; A61P 25/00; A61P 25/22; A61P 25/24; B05D 5/00

(52) U.S. Cl. 424/452; 514/438; 424/465; 427/2.21

(57) ABSTRACT

A delayed release dosage form comprising core comprising duloxetine or its pharmaceutically acceptable salts or derivatives thereof, optionally, other pharmaceutically acceptable excipient(s) thereof; intermediate layer; and enteric layer; wherein the dosage form comprises one/more dissolution enhancer(s), wherein the enteric layer comprises one/more enteric polymers other than hydroxypropylmethyl acetate succinate. A process of preparing a delayed release dosage comprising mixing pharmaceutically acceptable excipients with duloxetine or its pharmaceutically acceptable derivatives thereof; granulating the product of previous step compressing the granulate formed in previous step to form core, coating said core with intermediate layer followed by coating with one/more enteric polymers and optional finishing coating. A delayed release dosage form comprising: core comprising duloxetine or its pharmaceutically acceptable derivative thereof, intermediate layer and enteric layer comprising one/more enteric polymers other than hydroxypropylmethyl acetate succinate; wherein dosage form contains one/more dissolution enhancer(s) and has improved dissolution.

### FIELD OF THE INVENTION

The present invention provides delayed release compositions comprising duloxetine or its pharmaceutically acceptable salt, enantiomers, solvates, polymorphs or derivative thereof and one or more dissolution enhancer(s), wherein the composition has an improved dissolution and process of preparing the same.

### BACKGROUND OF THE INVENTION

Numerous active ingredients for example duloxetine suffer from the disadvantage of being poorly soluble in an aqueous medium, thus having an insufficient dissolution profile and, consequently, poor bioavailability, following oral administration. The therapeutic dose required to be administered must thus be increased in order to obviate this disadvantage.

Duloxetine hydrochloride (Duloxetine HCl) is a selective serotonin and norepinephrine reuptake inhibitor (“SSNRI”), having the chemical name (+)-(S)-N-methyl-γ-(1-naphthyloxy)-2-thiophenepropylamine hydrochloride.

Duloxetine hydrochloride is disclosed in U.S. Pat. No. 5,023,269, and is currently marketed by Eli Lilly for the treatment of major depressive disorder, general anxiety disorder, diabetic peripheral neuropathic pain and fibromyalgia under the trade name CYMBALTA® as 20, 30, and 60 mg delayed release enteric-coated capsules.

Delayed release formulations are advantageous, as they prevent exposure of an acid sensitive active pharmaceutical ingredient (“API”) to the acidic environment of a patient's stomach, preventing degradation of the API and/or irritation of the patient's stomach.

U.S. Pat. No. 5,508,276 (“the '276 patent”) discloses a delayed release duloxetine formulation in the form of an enteric duloxetine pellet. The disclosed enteric coating layer contains an enteric polymer having only a small number of carboxylic acid groups per repeating unit. Hydroxypropyl methyl cellulose acetate succinate (“HPMCAS”) is disclosed as the preferred enteric polymer. According to this patent, duloxetine was found to react with other enteric coatings to form a slowly soluble or insoluble coating leading to a disadvantageous drug-releasing profile and/or low bioavailability.

U.S. Patent Application 2006/0165776 discloses an enteric-coated composition and process for making the same but it does not address solubility issues.

U.S. Patent Application 2007/0292511 discloses an enteric-coated composition comprising inert beads layered with drug, which are further coated by an intermediate coating and then an enteric coating. The enteric coating layer contains enteric polymers other than HPMCAS, which do not require neutralization.

PCT application WO 2007/034503 discloses a controlled release dosage form of Duloxetine. The controlled release dosage forms of duloxetine in this invention are shown to lead to better safety profile and tolerability than the conventional delayed release formulations.

The inventors have now developed a delayed release dosage form of Duloxetine or its pharmaceutically acceptable salt thereof, which surprisingly exhibits, improved dissolution.

### OBJECTS OF THE INVENTION

It is an object of the invention is to provide a delayed release dosage form, which comprises a core comprising duloxetine or its pharmaceutically acceptable salt, enantiomers, polymorphs, solvates, hydrates or derivative thereof, an intermediate layer and an enteric layer comprising one or more enteric polymers other than hydroxypropylmethyl acetate succinate and one or more dissolution enhancer(s).

Another object of the invention is to provide a process of preparing a delayed release dosage form of comprising dissolution enhancer wherein the dosage form is manufactured comprising the steps of: (i) mixing pharmaceutically acceptable excipients with duloxetine or its pharmaceutically acceptable salt, enantiomers, polymorphs, solvates, hydrates or derivative thereof, (ii) granulating the product of step (i), (iii) compressing the granulate formed in step (ii) to form a core, (iv) coating the said core with an intermediate layer followed by (v) coating with one or more enteric polymers and an optional finishing coating which can be further filled into capsules.

Yet another object of the invention is to provide a delayed release dosage form comprising: a core comprising duloxetine or its pharmaceutically acceptable salt, enantiomers, polymorphs, solvates, hydrates or derivative thereof, an intermediate layer and an enteric layer comprising one or more enteric polymers other than hydroxypropylmethyl acetate succinate; wherein the dosage form contains one or more dissolution enhancer(s) and has an improved dissolution.

Another object of the invention is to provide dissolution profile of a delayed release dosage form of the invention comprising Duloxetine or its pharmaceutically acceptable salt, enantiomers, polymorphs, solvates, hydrates or derivative thereof, and one or more dissolution enhancer wherein the dosage form provides a dissolution rate (measured by the Ph. USP. Basket method at 100 rpm in 1000 ml 6.8 pH phosphate buffer at 37° C. and using UV detection at 290 nm) of about 20% to about 35% after 10 min, about 35% to about 55% after 15 min, about 55% to about 80% after 20 min, about 75% to about 95% after 30 min, about 85% to about 96% after 45 min, greater than 85% after 60 min.

Another object of the invention is to provide dissolution profile of a delayed release dosage form of the invention comprising salt, enantiomers, polymorphs, solvates, hydrates or derivative thereof, and one or more dissolution enhancer wherein the dosage form provides a dissolution rate (measured by the Ph. USP. Basket method at 100 rpm in 1000 ml 5.5 pH sodium phosphate buffer at 37° C. and using UV detection at 290 nm) of about 0% to about 10% after 10 min, about 0% to about 20% after 15 min, about 15% to about 35% after 20 min, about 35% to about 60% after 30 min, about 60% to about 80% after 45 min, greater than about 75% after 60 min.

### SUMMARY OF THE INVENTION

The present invention provides a delayed release dosage form which comprises a core comprising duloxetine or its pharmaceutically acceptable salt, enantiomers, polymorphs, solvates, hydrates or derivative thereof, an intermediate layer and an enteric layer comprising one or more enteric polymers other than hydroxypropylmethyl acetate succinate and one or more dissolution enhancer(s), wherein the composition has an improved dissolution and process of preparing the same.

### BRIEF DESCRIPTION OF THE INVENTION

The present invention provides enteric compositions suitable for oral administration comprising therapeutically effective amount of Duloxetine and dissolution enhancers wherein the composition has an improved dissolution. The term “duloxetine” encompasses various pharmaceutical equivalent isomers, enantiomers, complexes, salts, hydrates, polymorphs, esters etc of duloxetine.

The term “improved dissolution” means improved dissolution from over dissolution of the formulation of the invention without dissolution enhancer and over prior art, from, delayed release formulation of Duloxetine marketed under the brand name of Cymbalta®.

The term “composition” or “formulation” includes but not limited to solutions and/or suspensions, dispersions, concentrates, ready mix, powders, granules, tablets, micro-tablets, capsules, pellets, comprising duloxetine or its pharmaceutically acceptable derivative thereof in a core and coated with intermediate layer/s followed by enteric layer.

The term “seal coating layer” is synonymous to various terms like separating layer, seal coating layer intermediate layer, barrier coating layer, and the like.

The term “delayed release formulation” is synonymous to enteric release, prolonged release or modified release formulation and the like.

The said medicament according to the present invention comprises a formulation substantially as herein described, and in particular a capsule or a tablet or micro-tablets or granules or pellets filled in capsule formulation, typically an enteric or delayed release capsule formulation substantially as hereinafter further described.

The core comprises duloxetine which may be mixed with dissolution enhancer and with other pharmaceutically acceptable excipients, into a homogeneous mixture, and further formed into granulates using a suitable binder. The granulate may be optionally compressed into minitablets.

The term “dissolution enhancer” or “Solubility enhancer” can be used alternately and means an agent that is capable of improving the solubility of the active ingredient. The dissolution enhancer may be present at between about 0.2% to about 3.0% w/w, preferably from about 1.8% to about 2.2% w/w with respect to weight of drug. Different techniques have been employed by scientists to improve solubility and ensure sink conditions such as use of large dissolution volume, removal of dissolved drug, pH changes and addition of surfactants or their combinations. Among aforementioned approaches, pH modification and surfactant addition appear to be the simplest and can be tailored to resemble GI fluid environment.

The solubility enhancer or dissolution enhancer can be used to provide any of the advantageous characteristics to the compositions, including: increased solubility of the active, improved dissolution of the active agent, improved solubilization of the active ingredient upon dissolution; enhanced absorption and/or bioavailability of the active ingredient, and improved stability both the physical and chemical, of the active ingredient.

The dissolution-enhancing agent may include, but are not limited to, one or more agents that inhibit crystal formation of the pharmaceutical; surfactant, solubilizer, complexing agent, and the like or mixtures thereof.

Examples of the agents that inhibit crystal formation of the pharmaceutical or otherwise act by complexation therewith include polyvinylpyrrolidone, polyethylene glycol (particularly PEG 8000), alpha, beta or delta cyclodextrins and other modified cyclodextrins, gelatin, maltodextrin, sorbitol, and polyglyceryl mixed vegetable fatty acid esters.

The term “surfactant” is used in its conventional sense in this invention. Any surfactant is suitable, whether it be amphoteric, non-ionic, cationic or anionic.

Examples of such surfactants are: sodium lauryl sulfate, monooleate, monolaurate, monopalmitate, monostearate or another ester of polyoxyethylene sorbitane, sodium dioctylsulfosuccinate (DOSS), lecithin, stearylic alcohol, cetostearylic alcohol, cholesterol, polyoxyethylene ricin oil, polyoxyethylene fatty acid glycerides, Poloxamer®, etc. Mixtures of surfactants are also suitable. A high HLB, micelle-forming surfactant includes non-ionic and/or anionic surfactants and selected from Tween 20, Tween 60 or Tween 80, Gelucire 44/14, and Labrasol; polyoxyethylene or polyethylene-containing surfactants, or other long chain anionic surfactants.

Alternately, the said core is comprised of duloxetine mixed with solubility enhancer and with other pharmaceutically acceptable excipients and directly compressed together. The compressed core may be in form of granules, or the granules may further be compressed together into minitablets.

The mixing here may be purely physical mixing, deposition, coating, adsorption, aggregation or adhesion and alike. Such a mixing of Duloxetine and other pharmaceutically acceptable ingredients may be achieved in several different ways. According to one of the embodiment of the invention, such a mixing is achieved by granulation, and preferably through fluidized bed granulation.

The other pharmaceutically acceptable excipients may include diluents, binders, disintegrating agents, lubricants and the like.

Diluents include, but are not limited to, calcium phosphate-dibasic, cellulose-microcrystalline, cellulose powdered, calcium silicate, mannitol, sorbitol, xylitol, maltitol, sucrose, lactose, starch and combination thereof.

Binders include, but not limited to, such as hydroxypropylmethylcellulose, hydroxypropylcellulose, polyvinylpyrrolidone, methylcellulose, acacia and gelatin or mixtures thereof.

Disintegrating agents are selected from, but are not limited to, croscarmellose sodium, crospovidone, sodium starch glycolate, sodium carboxymethyl cellulose, hydroxypropyl cellulose, alginic acid, alginates, polacrilin potassium or combination thereof.

Lubricants include, but not limited to, such as magnesium stearate, crospovidone, beta lactose, sodium stearylfumarate, hydrogenated vegetable oil, stearic acid, calcium stearate, glyceryl behenate, sodium lauryl sulfate, talc and finely divided silicon dioxide or mixtures thereof.

The said core is further coated with intermediate layer followed by coating with enteric material and optionally with a finishing layer.

The functions of the intermediate layer is to provide a smooth base for the application of the enteric layer, to prolong resistance to acid conditions, to improve stability by inhibiting any interaction between the drug and the enteric polymer in the enteric layer, and to improve stability by protecting the drug from light exposure.

The intermediate layer is preferably composed of a substance (or a mixture of such substances) that does not react or affect the stability of the core; comprising duloxetine or its pharmaceutically acceptable derivative thereof nor adversely affects bioavailability or release of the Duloxetine or its pharmaceutically acceptable salts thereof. Typical examples of such substances that can be used in the intermediate layer include coating agents such as organic or inorganic polymers, sugars, celluloses and cellulose derivatives such as hydroxypropylmethyl cellulose, hydroxypropylcellulose, hydroxypropylethyl cellulose, ethylcellulose and the like.

In one embodiment of the invention, the intermediate layer comprises at least one substance selected from a group comprising of pharma grade sugar, mannitol, talc, and aerosil. The sub coating layers may optionally contain one or more pharmaceutically acceptable excipients such as sodium lauryl sulfate, talc, polyethylene glycol, propylene glycol polysorbate, calcium carbonate, sodium carbonate, silicon dioxide, magnesium oxide, silica anhydride, calcium silicate, magnesium hydroxide, magnesium carbonate, aluminium hydroxide, calcium stearate and magnesium stearate, sodium stearylfumarate and the like and coloring agents such as iron oxide yellow or red. It is an advantageous feature of this invention that the intermediate layer may also contain solubility enhancers. The intermediate layer may be applied to the core using any known technique. These techniques include with any limitation for example powder coating, spraying, pan coating, and alike.

The enteric layer is applied to get delayed release of the duloxetine hydrochloride. Preferably the enteric layer is substantially insoluble in acidic environments, such as stomach, but is soluble in near-neutral environments, such as small intestine. The formulation remains intact as it passes through the acidic environment in the stomach, but dissolves and releases the duloxetine hydrochloride once it passes into the near neutral environment of the small intestine. The enteric layer preferably contains a polymer that dissolves at a pH above about 5.5.

The enteric layer is comprised of an enteric material selected from cellulose acetate phthalate, hydroxypropyl methylcellulose phthalate, polymethacrylates, polyvinylacetate phthalate, and acrylic acid polymers such as Eudragit and the like or a combination there of provided it is free of hydroxypropylmethylcellulose acetyl succinate. The enteric coating layer may additionally contain solubility enhancers, plasticizer and/or conventional pharmaceutical excipients used to facilitate coating such as sodium lauryl sulfate, talc, colloidal silica, sodium stearylfumarate and the like. The plasticizers include triethyl citrate, polysorbate 80, triacetin and the like. The coating aids include, for example, fatty acid glycerol esters, polyethylene glycol.

Enteric polymers may be applied as coating from aqueous suspensions or from solutions in aqueous or organic solvents. Typical solvents which may be used to apply the subcoating layer and acid resistant enteric coating layer include water, isopropyl alcohol, ethanol, acetone, methylene chloride.

The finishing layer is an optional one, and is preferably applied to aid in the handling of the formulation. The enteric coating may attain some electrostatic charges, which may result in the sticking of the formulation to the packaging; the finishing layer prevents the coating from coming into contact with the packaging, thereby avoiding this problem. The optional finishing layer comprises a coating agent and, optionally one or more pharmaceutically acceptable excipients. Preferably, the coating agent is hypromellose. The additional pharmaceutically acceptable excipients may include excipients commonly used in pharmaceutical formulations for use in finish layers or coatings. Preferably, the additional pharmaceutically acceptable excipients are selected from thickening agents, glidants, and coloring agents. More preferably, the additional pharmaceutically acceptable excipients are selected from talc, colloidal silicon dioxide, and titanium dioxide.

The invention also encompasses a process for the preparation of duloxetine hydrochloride delayed release formulation, comprising a core comprising duloxetine hydrochloride; a seal coating layer; an enteric layer comprising hydroxypropyl methyl cellulose phthalate and/or a methacrylic acid copolymer; and then, optionally, a finishing layer.

In another embodiment the delayed release formulation of the present invention comprises an inert nuclei coated with a drug layer followed by coating with separating layer and an enteric layer and/or a finishing layer.

Preferably, each layer is applied in the form of a suspension and/or a solution, and, more preferably, each layer is spray coated. Preferably, each layer is dried prior to the application of the next successive coating.

Once prepared, the formulation may be packed into a solid pharmaceutical dosage form such as a tablet or a capsule. Preferably the formulation is filed into a capsule.

The invention is not limited to the particular embodiments of the invention described below, as variations of the particular embodiments may be made and still fall within the scope of the invention. It is also to be understood that the terminology employed is for the purpose of describing particular embodiments, and is not intended to be limiting.

### EXAMPLES

### Example 1

### Mini Tablets Filled in Capsules

| Ingredients | Quantity per Tablet in mg (60 mg) | |
|---|---|---|
| Core | | |
| Duloxetine Hydrochloride | 5.66 | |
| Croscarmellose Sodium | 2.00 | |
| Lactose | 10.44 | |
| Pregelatinised starch | 0.40 | |
| Purified Water | Q.S. | |
| Magnesium stearate | 0.5 | |
| Average Weight | 19 | mg |
| Seal coating | | |
| Hydroxypropylmethylcellulose | 2.28 | |
| Isopropyl Alcohol | Q.S. | |
| Dichloromethane | Q.S. | |
| Average Weight | 21.28 | mg |
| Enteric coating | | |
| Hypromellose phthalate (HPMCP-55) | 1.422 | |
| Triethyl citrate | 0.142 | |
| Talc | 0.355 | |
| Dichloro methane | Q.S. | |
| Methanol | Q.S. | |
| Final Average. Weight | 23.2 | mg |

### Procedure:

1. Sift Duloxetine HCl; lactose monohydrate and Croscarmellose Sodium through suitable sieve.
2. Disperse Starch in purified water and prepare a binder solution.
3. Granulate step 1 with the binder solution of Step 2.
4. Dry the granules and sift
5. Lubricate Step 4 with Croscarmellose Sodium and Magnesium Stearate.
6. Compress Step 5
7. Seal Coat and Enteric coat the tablets using the coating composition as in Table above.
8. Fill enteric-coated mini tablets in empty gelatin capsules.

### Example 2

### Mini Tablets Filled in Capsules

| Ingredients | Quantity per Tablet in mg (60 mg) | |
|---|---|---|
| Core | | |
| Duloxetine Hydrochloride | 5.66 | |
| Croscarmellose Sodium | 2.00 | |
| Lactose | 10.377 | |
| Polysorbate 80 | 0.40 | |
| Pregelatinised starch | 0.063 | |
| Purified Water | Q.S. | |
| Magnesium stearate | 0.50 | |
| Average Weight | 19 | mg |
| Seal coating | | |
| Hydroxypropylmethylcellulose | 2.28 | |
| Isopropyl Alcohol | Q.S. | |
| Dichloromethane | Q.S. | |
| Average Weight | 21.28 | mg |
| Enteric coating | | |
| Hypromellose phthalate (HPMCP-55) | 1.422 | |
| Triethyl citrate | 0.142 | |
| Talc | 0.355 | |
| Dichloro methane | Q.S. | |
| Methanol | Q.S. | |
| Final Average. Weight | 23.2 | mg |

### Example 3

### Mini Tablets Filled in Capsules

| Ingredients | Quantity per Tablet in mg (60 mg) | |
|---|---|---|
| Core | | |
| Duloxetine Hydrochloride | 5.66 | |
| Croscarmellose Sodium | 2.00 | |
| Lactose | 10.377 | |
| Polysorbate 80 | 0.40 | |
| Pregelatinised Starch | 0.063 | |
| Purified Water | Q.S. | |
| Magnesium stearate | 0.50 | |
| Average Weight | 19 | mg |
| Seal coating | | |
| Hydroxypropylmethylcellulose | 2.26 | |
| Polysorbate 80 | 0.02 | |
| Isopropyl Alcohol | Q.S. | |
| Dichloromethane | Q.S. | |
| Average Weight | 21.28 | mg |
| Enteric coating | | |
| Hypromellose phthalate (HPMCP-55) | 1.422 | |
| Triethyl citrate | 0.142 | |
| Talc | 0.355 | |
| Dichloro methane | Q.S. | |
| Methanol | Q.S. | |
| Final Average. Weight | 23.2 | mg |

### Example 4

### Mini Tablets Filled in Capsules

| Ingredients | Quantity per Tablet in Mg (60 mg) | |
|---|---|---|
| Core | | |
| Duloxetine Hydrochloride | 5.66 | |
| Croscarmellose Sodium | 2.00 | |
| Lactose monohydrate | 10.377 | |
| Pregelatinised Stacrh | 0.40 | |
| Polysorbate 80 | 0.063 | |
| Purified Water | Q.S. | |
| Magnesium stearate | 0.50 | |
| Average Weight | 19 | mg |
| Seal coating | | |
| Hydroxypropylmethylcellulose | 2.26 | |
| Polysorbate 80 | 0.02 | |
| Isopropyl Alcohol | Q.S. | |
| Dichloromethane | Q.S. | |
| Average Weight | 21.28 | mg |
| Enteric coating | | |
| Hypromellose phthalate (HPMCP-55) | 1.42 | |
| Triethyl citrate | 0.14 | |
| Talc | 0.355 | |
| Polysorbate 80 | 0.02 | |
| Dichloro methane | Q.S. | |
| Methanol | Q.S. | |
| Final Average Weight | 23.2 | mg |

### Procedure:

1. Sift Duloxetine HCl; lactose monohydrate and Croscarmellose Sodium through suitable sieve.
2. Dissolve Tween 80 in Purified water and disperse Starch in it and prepare a binder solution.
3. Granulate step 1 with the binder solution of Step 2.
4. Dry the granules and sift
5. Lubricate Step 4 with Croscarmellose Sodium and Magnesium Stearate.
6. Compress Step 5
7. Seal Coat and Enteric coat the tablets using the coating composition as in Table above.
8. Fill enteric-coated mini tablets in empty gelatin capsules.

### Dissolution Study

A comparative dissolution was carried out of a delayed release capsules containing about 60 mg Duloxetine Hydrochloride prepared according to example 1 containing no surfactant with a delayed release capsules containing about 60 mg Duloxetine Hydrochloride prepared according to example 4 containing a surfactant, such as Polysorbate 80 (polyoxyethylene sorbitane mono-oleate). This surfactant is the object of a monograph in the Pharmacopoeias, and is thus easy to implement (being also a water-soluble liquid product), but other surfactants can also be used.

The USP apparatus is used under the following conditions: volume of medium: 1000 ml; medium temperature: 37° C.±5° C.; rotation speed: 100 rpm; samples taken: at 10, 15, 20 and 30 minutes. Determination of the amount dissolved is carried out by spectrophotometry. Dissolution is carried out first in 0.1NHCl for 2 hrs followed by 6.8-pH phosphate buffer for enteric-coated formulation.

TABLE 1
Dissolution of core of Minitablets in 6.8 pH Phosphate Buffer

% of Drug released

| Sampling Time in Min | Example 1 | Example 2 |
|---|---|---|
| 10 | 66.90 | 92.20 |
| 15 | 69.70 | 98.80 |
| 20 | 70.00 | 101.30 |
| 30 | 71.50 | 102.80 |

TABLE 2
Dissolution of Seal coated core of Minitablets
in 6.8 pH Phosphate Buffer

% of Drug released

| Sampling Time in Min | Example 2 | Example 3 |
|---|---|---|
| 10 | 63.60 | 92.70 |
| 15 | 74.40 | 100.00 |
| 20 | 82.80 | 100.60 |
| 30 | — | 102.50 |

TABLE 3
*Dissolution of Seal coated core of Minitablets over-coated
with Enteric Coating in 6.8 pH Phosphate Buffer

| | % of Drug released | | |
|---|---|---|---|
| Sampling Time in Min | Example 1 | Example 3 | Example 4 |
| 10 | 17.0 | 19.0 | 26.70 |
| 15 | 27.2 | 35.1 | 42.80 |
| 20 | 39.60 | 59.8 | 71.00 |
| 30 | 55.20 | 72.3 | 90.70 |
| 45 | 68.3 | 85.6 | 97.10 |
| 60 | — | — | 98.20 |
| 90 | — | — | 99.80 |

*Dissolution is carried out first in 0.1N HCl for 2 hrs followed by 6.8-pH phosphate buffer

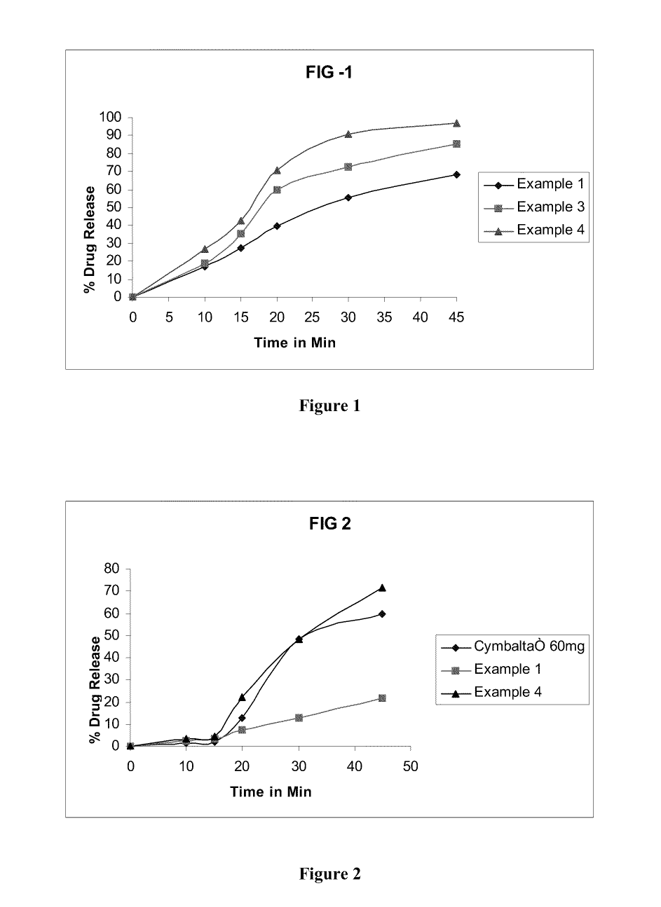

The dissolution results obtained at 6.8-pH phosphate buffer depicted in table 3 are shown graphically in FIG. 1, on which the percentage of dissolution is shown.

These results clearly show that the compositions prepared according to example 4 have a dissolution profile, which is distinctly better than that of the compositions prepared according to examples 1, 2 & 3.

A comparative dissolution was carried out of a delayed release capsules containing about 60 mg Duloxetine Hydrochloride prepared according to example 1 containing no surfactant with a delayed release capsules containing about 60 mg Duloxetine Hydrochloride prepared according to example 4 containing a surfactant, such as Polysorbate 80 (polyoxyethylene sorbitane mono-oleate). Comparison was also made with prior art formulation which being delayed release 60 mg capsules of Duloxetine HCl commercialized under the brand name Cymbalta®.

A dissolution medium, which is discriminating, in other words one in which two products having very different dissolution profiles in gastrointestinal fluids and having very different dissolution curves, is looked for.

The USP apparatus is used under the following conditions: volume of medium: 1000 ml; medium temperature: 37° C.±5° C.; rotation speed: 100 rpm; samples taken: at 10, 15, 20, 30, 45, 60 and 90 minutes. Determination of the amount dissolved is carried out by spectrophotometry. Dissolution is carried out first in 0.1N HCl for 2 hrs followed by 5.5 pH sodium phosphate buffer.

TABLE 4
Dissolution of Seal coated core of Minitablets over-coated
with Enteric Coating in 5.5 pH Sodium Phosphate Buffer

| | % of Drug released | | |
|---|---|---|---|
| Sampling Time in Min | Cymbalta ® 60 mg | Example 1 | Example 4 |
| 10 | 1.6 | 2.3 | 3.4 |
| 15 | 1.9 | 3.4 | 4.5 |
| 20 | 13 | 7.6 | 22.3 |
| 30 | 48.3 | 12.9 | 48.6 |
| 45 | 59.9 | 21.8 | 71.6 |
| 60 | 76.9 | — | 83.3 |
| 90 | 84.2 | — | 101.7 |

Surprisingly, the results show that the compositions according to example 4 have a dissolution profile, which is distinctly better than that of the composition prepared according to examples 1.

The results also show that the compositions of the present invention have a dissolution profile that is an improvement over compositions of the prior art and may lead to a considerably enhanced bioavailability of the active ingredient compared to that obtained with compositions of the prior art.

The dissolution results obtained at 5.5 pH sodium phosphate buffer depicted in table 4 are shown graphically in FIG. 2, on which the percentage of dissolution is shown.

1. A delayed release dosage form comprising
a core comprising duloxetine or its pharmaceutically acceptable salts or derivatives thereof, optionally, other pharmaceutically acceptable excipient(s) thereof;
an intermediate layer; and
an enteric layer;
characterized in that the dosage form comprises one or more dissolution enhancer(s),
wherein the enteric layer comprises one or more enteric polymers other than hydroxypropylmethyl acetate succinate.

2. A delayed release dosage form according to claim 1, wherein the core comprises inert nuclei coated with a drug layer comprising Duloxetine or its pharmaceutically acceptable salt or derivatives thereof.

3. A delayed release dosage form according to claim 1, wherein one or more dissolution enhancer(s) is present in one or more portions of the dosage form.

4. A delayed release dosage form according to claim 1, wherein the dissolution enhancer is selected from the group comprising agents that inhibit crystal formation of the pharmaceutical, complexing agents and surfactants.

5. A delayed release dosage form according to claim 4, wherein the dissolution enhancer is a surfactant.

6. A delayed release dosage form according to claim 5, wherein the dissolution enhancer is polysorbate 80.

7. A delayed release dosage form according to claim 1, wherein the dissolution enhancer is from about 0.2% to about 3.0% by weight of the active ingredient.

8. A delayed release dosage form according to claim 7, wherein the unit dosage is a capsule or a tablet.

9. A delayed release dosage form according to claim 1, wherein the dosage form is selected from pellets, granules, minitablets, caplets, tablets and capsules.

10. A delayed release dosage form according to claim 1, further comprises pharmaceutically acceptable excipients selected from diluents, binders, disintegrating agents and lubricants.

11. A delayed release dosage form according to claim 1, wherein the intermediate layer comprises a coating agent and additional pharmaceutically acceptable excipients selected from diluents, anti-adherents, thickening agents, plasticizers and dissolution enhancers.

12. A delayed release dosage form according to claim 1, wherein the enteric layer comprises enteric coating agents other than hydroxypropylcelluloseacetyl succinate and pharmaceutically acceptable excipients selected from glidants, plasticizers and dissolution enhancers.

13. A process of preparing a delayed release dosage form according to claim 1, wherein the process comprises the steps of: (i) mixing pharmaceutically acceptable excipients with duloxetine or its pharmaceutically acceptable derivatives thereof; (ii) granulating the product of step (i), (iii) compressing the granulate formed in step (ii) to form a core, (iv) coating the said core with an intermediate layer followed by (v) coating with one or more enteric polymers and an optional finishing coating.

14. A process of preparing a delayed release dosage form according to claim 13, wherein the dosage form is further filled into capsule.

15. A delayed release dosage form comprising: a core comprising duloxetine or its pharmaceutically acceptable derivative thereof, an intermediate layer and an enteric layer comprising one or more enteric polymers other than hydroxypropylmethyl acetate succinate; wherein the dosage form contains one or more dissolution enhancer(s) and has an improved dissolution.

16. A delayed release dosage form according to claim 15, wherein the dosage form provides a dissolution rate (measured by the Ph. USP. Basket method at 100 rpm in 1000 ml 6.8 pH phosphate buffer at 37° C. and using UV detection at 290 nm) of about 20% to about 35% after 10 min, about 35% to about 55% after 15 min, about 55% to about 80% after 20 min, about 75% to about 95% after 30 min, about 85% to about 96% after 45 min, greater than 85% after 60 min.

17. A delayed release dosage form as in claim 15, wherein the dosage form provides a dissolution rate (measured by the Ph. USP. Basket method at 100 rpm in 1000 ml 5.5 pH sodium phosphate buffer at 37° C. and using UV detection at 290 nm) of about 0% to about 10% after 10 min, about 0% to about 20% after 15 min, about 15% to about 35% after 20 min, about 35% to about 60% after 30 min, about 60% to about 80% after 45 min, greater than about 75% after 60 min.
